(19) United States
(12) Patent Application Publication
(10) Pub. No.: US 2009/0149531 A1
(43) Pub. Date: Jun. 11, 2009

(54) CHEMICAL COMPOSITION DERIVED FROM P-HYDROXYPHENYL PROPIONIC ACID FOR THE TREATMENT OF PSORIASIS

(75) Inventors: Juan Pablo PIVEL RANIERI, Madrid (ES); Juan Manuel Ferrer Cuesta, Madrid (ES); Fernando Martinez Galan, Navarra (ES); Juan Manuel Irache, Navarra (ES); Jose Luis Novella Robisco, Madrid (ES); Juan Jose Perez Rueda, Madrid (ES); Ma Paz Maria Martin, Madrid (ES); Julio Alvarez-Builla Gomez, Madrid (ES); Jaime Bermejo, Tenerife (ES); Fernando Vidal Vanaclocha, Lieona Vizcaya (ES)

Correspondence Address: EGBERT LAW OFFICES, 412 MAIN STREET, 7TH FLOOR, HOUSTON, TX, 77002, US

(73) Assignee: APOTEKNOS PARA LA PIEL, S.L., Madrid (ES)

(21) Appl. No.: 12/332,324

(22) Filed: Dec. 10, 2008

(30) Foreign Application Priority Data
Dec. 11, 2007 (EP) 07 380 346.2

(51) Int. Cl. A61K 31/341; C07D 307/04; A61P 17/00

(52) U.S. Cl. 514/461; 549/323

(57) ABSTRACT

The present invention is a chemical composition including:

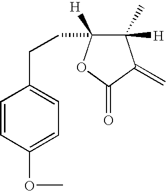

Use of the compound with the chemical formula can be applied in pharmaceutical compositions for the treatment of psoriasis.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to the use of compound A (derived from P-hydroxyphenyl propionic acid):

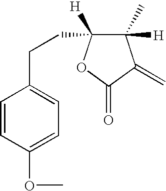

Compound A

for the treatment of psoriasis.

2. Description of Related Art Including Information Disclosed Under 37 CFR 1.97 and 37 CFR 1.98

Psoriatic skin is characterized by the hyperproliferation of keratinocytes, inflammation, angiogenisis and dilation of the blood vessels. There are different types of treatments on the market to prevent or reduce this type of skin disorder that is prevalent in 2% of the world's population, such as the use of topical creams with certain compositions such as those disclosed in the prior art ES2214105, ES2231007, ES2188426 and ES2186586, or specific products such as derivatives of vitamin D and retinoic acid, and certain corticosteroids. There are also many publications that show the great social impact of this disorder (“Mechanism of psoriasis” Frank O. Nestle, Curdin Conrad, Drug Discovery Today: Disease Mechanisms Vol. I No. 3 2004) and the importance of finding compounds to help in its treatment and enable its elimination (“Dermatology Online Journal” Volume 6 No. 1 Steven Feldman, M.D., PhD).

Most products for psoriasis that are known at present are derived from the use of products initially developed for other pathologies such as rheumatoid arthritis, inflammatory bowel disease, etc. and they are very aggressive treatments, or strong immunosuppressants such as cyclosporine, rapamycin and methotrexate, or very expensive treatments such as anti-TNF monoclonal antibodies, which cannot be used as chronic therapies and have serious side effects.

Calcitriol (1-alpha,25-dihydroxycholecalciferol) is the active form of vitamin D that is found in the body (vitamin D₃) and its use is known for the treatment of psoriasis, as is disclosed in various publications and patents such as “Calcitriol ointment and clobetasol propionate cream: a new regimen for the treatment of plaque psoriasis” European Journal of Dermatology, Vol. II, No. 3, May 2003 (2003-05) pp. 261-265, WO2006008354 or U.S. Pat. No. 4,610,978.

In an attempt to find new compositions that make it possible to treat this disorder, it has surprisingly been discovered that compound A has the following important characteristics:
1) Safe and effective compound that does not present the side effects of vitamin D derivatives.
2) Low permeability, which makes it possible to define the product as topical and enables the mutagenesis test (Ames test) to be performed without hepatic metabolisation, as the product is not absorbed topically.
3) It is an active topical product that does not cause toxic effects and is therefore a safe product.
4) Its antipsoriatic activity is similar to that of calcitriol.

### BRIEF SUMMARY OF THE INVENTION

The present invention is the use of a chemical composition of compound A:

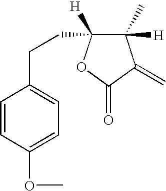

### BRIEF DESCRIPTION OF THE SEVERAL VIEWS OF THE DRAWINGS

FIG. 1 is a graph illustration of concentration vs. time, involving a study of the chemical composition of the present invention.

FIG. 2 is a graph illustration of concentration vs. time, involving the same study of the chemical composition of the present invention.

### DETAILED DESCRIPTION OF THE INVENTION

Various studies have been carried out to show the benefits of using compound A:

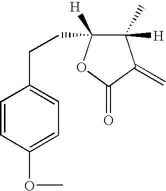

to treat psoriasis.

1. Permeability Study

Skin from pig ears was used as the experimental system to which a hydroalcoholic solution of compound A 6.36×10⁻² mg/ml and 6.36×10⁻³ mg/ml was applied, the reference product being a control hydroalcoholic solution (without compound A). For this study 4 different hydroalcoholic solutions were prepared. The first included 6.36 μg/ml of compound A, the second included 63.6 μg/ml, and the third and fourth were control solutions without compound A.

All the preparations were tested on the pig ear skin experimental system. Samples were taken in the diffuser compartment at 6 different times (0, 1, 2, 6, 12 and 24 hours). All the samples (recipients) were quantified by HPLC.

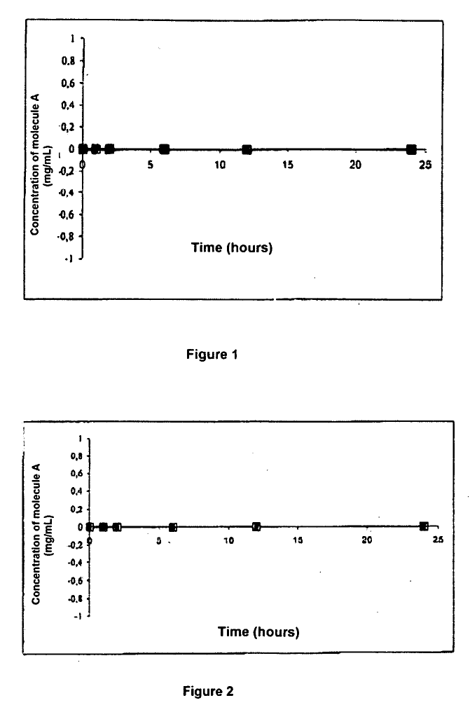

FIGS. 1 and 2 show the amount of compound A collected in the recipient:

FIG. 1. Concentration of compound A in the recipient of the Franz cell for a sample with a concentration of 63.6 μg/ml of A.

FIG. 2. Concentration of compound A in the recipient of the Franz cell for a sample with a concentration of 6.36 μg/ml of A.

As can be observed, in every case the amount of compound A dissolved in the recipient was lower that 0.01 mg/ml (quantification limit of the HPLC technique). Moreover, in no case could any sign be observed that would make it possible to predict the passage of a minimum fraction of molecule A. Therefore, is can be stated that in the conditions used in the study there is no transdermal flow of compound A through the skin of the pig ear.

In conclusion, it can be said that molecule A, in the formulated hydroalcoholic vehicle, does not cross the pig's skin in the conditions tested, at least in a sufficient amount to be detected and quantified using the proposed analytical technique.

2. Ames Test

The mutagenic activity of compound A was evaluated using the Ames test without external metabolisation. The Ames test is based on the use of bacterial strains to detect mutations in the DNA in vitro using bacterial strains that make it possible to detect types of damage to the genome. As the product is defined as topical, since it is not permeable as is shown in the previous test, the mutagenisis test (Ames test) is performed without hepatic metabolisation, as the topical product is not absorbed.

Bacteria of 4 strains of Salmonella typhimurium that are internationally recommended for this test, TA1535, TA1537, TA98 and TA100, were used. The test was carried out according to highly standardised protocols. Three concentrations of compound A were tested, being seeded on 3 plates for each concentration in a single experiment. The test was carried out without external metabolic activation. The plates were incubated at 37° C. and the reverting colonies were counted after 48 hours.

The results are as follows:

TABLE 1
Salmonella typhimurium TA98

| | Coloniesper | | | | | | |
|---|---|---|---|---|---|---|---|
| | plate | | | | | | |
| | 1 | 2 | 3 | 4 | Mean | SD | Mutagenic Ind, |
| H−/B− | 0 | 0 | 0 | 0 | 0 | 0 | 0 |
| H+/B+ | 8 | 11 | 12 | 7 | 9.5 | 2.38047614 | 1 |
| H+/B+ DMSO | 10 | 13 | 10 | 10 | 10.75 | 1.5 | 1.131578947 |
| H+/B+ Comp, A 10 μg/ml | 9 | 6 | 12 | 14 | 10.25 | 3.5 | 1.078947368 |
| H+/B+ Comp, A 100 μg/ml | 15 | 12 | 8 | 12 | 11.75 | 2.87228132 | 1.236842105 |
| H+/B+ Comp, A 1 mg/ml | 8 | 4 | 6 | 17 | 8.75 | 5.73730483 | 0.921052632 |
| H+/B+ 4-Nitro-0-Phe . . . (2.5 μg/plate) | 29 | 29 | 17 | 22 | 24.25 | 5.85234996 | 2.552631579 |

TABLE 2
Salmonella typhimurium TA100

| | Colonies per | | | | | | |
|---|---|---|---|---|---|---|---|
| | plate | | | | | | |
| | 1 | 2 | 3 | 4 | Mean | SD | Mutagenic Ind, |
| H−/B− | 0 | 0 | 0 | 0 | 0 | 0 | 0 |
| H+/B+ | 66 | 55 | 35 | 38 | 48.5 | 14.6173413 | 1 |
| H+/B+ DMSO | 31 | 28 | 52 | 43 | 38.75 | 10.7819293 | 0.798969072 |
| H+/B+ Comp, A 10 μg/ml | 38 | 41 | 44 | 54 | 44.25 | 6.94622199 | 0.912371134 |
| H+/B+ Comp, A 100 μg/ml | 45 | 35 | 31 | 51 | 40.5 | 9.14694849 | 0.835051546 |
| H+/B+ Comp, A 1 mg/ml | 52 | 37 | 38 | 40 | 44 | 7.61577311 | 0.907216495 |
| H+/B+ 4-Nitro-0-Phe . . . (2.5 μg/plate) | 94 | 72 | 74 | 87 | 81.75 | 10.5316982 | 1.68556701 |

TABLE 3
Salmonella typhimurium TA1535

| | Colonies per | | | | | | |
|---|---|---|---|---|---|---|---|
| | plate | | | | | | |
| | 1 | 2 | 3 | 4 | Mean | SD | Mutagenic Ind, |
| H−/B− | 0 | 0 | 0 | 0 | 0 | 0 | 0 |
| H+/B+ | 0 | 4 | 3 | 1 | 2 | 1.82574186 | 1 |
| H+/B+ DMSO | 5 | 1 | 1 | 0 | 7.75 | 2.21735578 | 0.875 |
| H+/B+ Comp, A 10 μg/ml | 1 | 2 | 1 | 0 | 1 | 0.81649658 | 0.5 |
| H+/B+ Comp, A 100 μg/ml | 1 | 2 | 1 | 0 | 1 | 0.81649658 | 0.5 |
| H+/B+ Comp, A 1 mg/ml | 1 | 0 | 2 | 1 | 1 | 0.81649658 | 0.5 |
| H+/B+ 4-Nitro-0-Phe . . . (2.5 μg/plate) | 70 | 102 | 103 | 76 | 87.75 | 172119145 | 43.875 |

TABLE 4
Salmonella typhimurium TA1537

| | Colonies per | | | | | | |
|---|---|---|---|---|---|---|---|
| | plate | | | | | | Mutagenic |
| | 1 | 2 | 3 | 4 | Mean | SD | Ind, |
| H−/B− | 0 | 0 | 0 | 0 | 0 | 0 | 0 |
| H+/B+ | 0 | 1 | 0 | 1 | 0.5 | 0.57735027 | 1 |
| H+/B+ DMSO | 1 | 1 | 1 | 1 | 1 | 0 | 2 |
| H+/B+ Comp, | 0 | 1 | 0 | 0 | 0.25 | 0.5 | 0.5 |
| A 10 μg/ml | | | | | | | |
| H+/B+ Comp, | 0 | 1 | 0 | 2 | 0.75 | 0.95742711 | 1.5 |
| A 100 μg/ml | | | | | | | |
| H+/B+ Comp, | 0 | 0 | 0 | 0 | 0 | 0 | 0 |
| A 1 mg/ml | | | | | | | |
| H+/B+ 4-Nitro-0- | 74 | 135 | 46 | 56 | 77.75 | 39.8862967 | 155.5 |
| Phe . . . | | | | | | | |
| (2.5 μg/plate) | | | | | | | |

As can be observed, there was no increase in the dose according to the reversion frequency per colony for any of the Salmonella strains that were tested, which is the mutagenicity indicator for this test. It can therefore be concluded that the results were negative in all the tested strains of Salmonella typhimurium (TA1535, TA1537, TA98 and TA100), and compound A does not induce mutagenic effects at the concentrations evaluated.

3. Evaluation of Antipsoriatic Activity

The activity of compound A on cell hyperproliferation in hairless rats was evaluated using the model of desquamation in hairless rats described in the publication: Bräutigam, M., Hübner, H., Rach, P., Thieroff-Ekerdi, R. “Effects of Calcipotriol (MC 903) and Calcitriol after Topical Application on the Skin of Hairless Rats”, Skin Pharmacol 1992; 5:87-92.

To do this Calcitriol was used as the reference product at 0.01% and compound A was used as the test product at 1% and 0.1% (100 μg/cm² and 10 μg/cm², respectively). A Control Group was included, administered only with the vehicle (absolute ethanol).

The animals were randomly distributed into the following experimental groups:

TABLE 5
Experimental Group Distribution

| | | | | Volume of | Route of |
|---|---|---|---|---|---|
| Treatment | Dose | n | Group | administration | administration |
| Control | | | A | | Topical |
| Calcitriol | 1 μg/cm² | 6 | B | 100 μl/10 cm² | |
| 0.01% | | | | | |
| Comp. A | 100 μg/cm² | 6 | C | 100 μl/10 cm² | |
| 1% | | | | | |
| Comp. A | 10 μg/cm² | 6 | D | 100 μl/10 cm² | |
| 0.11% | | | | | |

The treatments were administered topically, once a day for 10 days (100 μg/side/animal). The animals were weighed on a daily basis throughout the study and the grade of erythema was evaluated (0=Absence of erythema, 1=Low grade erythema, 3=High grade erythema). On the last day of the study, day 11, the amount of corneous material extracted from the epidermis of the animals was evaluated.

Tables 6, 7 and 8 show the overall values corresponding to the amount of corneous material extracted, the mean evaluation of erythema and the body weight of the animals, respectively:

TABLE 6
Overall evaluation of the amount of corneous material extracted

| | | | | | Total sum of the right and left flanks (mg) | | | | | |
|---|---|---|---|---|---|---|---|---|---|---|
| | | | Route of | Volume of | Portal | Portal | Portal | Portal | Portal | Portal |
| Group | Treatment | n | administration | administration | No, 1 | No, 2 | No, 3 | No, 4 | No, 5 | No, 6 |
| A | Control | 6 | Topically | 0.2 mL/animal | 3.3 ± 0.21 | 2.4 ± 0.64 | 1.0 ± 0.12 | 0.8 ± 0.13 | 0.83 ± 0.23 | 8.2 ± 0.81 |
| B | Coleitriol | 6 | Topically | 0.2 mL/animal | 13.5 ± 4.89 | 7.7 ± 1.43 | 5.9 ± 0.94 | 4.5 ± 0.60 | 3.7 ± 0.35 | 21.7 ± 2.07 |
| | 0.01% | | | | | | | | | |
| C | Compound A | 6 | Topically | 0.2 mL/animal | 3.1 ± 0.75 | 2.7 ± 0.23 | 2.0 ± 0.26 | 1.4 ± 0.169 | 1.4 ± 0.23 | 12.6 ± 0.76 |
| | 1% | | | | | | | | | |
| D | Compound A | 6 | Topically | 0.2 mL/animal | 3.1 ± 0.17 | 1.7 ± 0.08 | 1.1 ± 0.14 | 1.1 ± 0.22 | 1.1 ± 0.28 | 8.1 ± 0.47 |
| | 0.1% | | | | | | | | | |

TABLE 7
Mean evaluation of erythema

| | | | Route of | Volume of | Assessment of erythema | | | |
|---|---|---|---|---|---|---|---|---|
| Group | Treatment | n | administration | administration | Baseline | Day 2 | Day 3 | Day 4 |
| A | Control | 6 | Topically | 0.2 mL/animal | 0.0 ± 0.00 | 0.0 ± 0.00 | 0.0 ± 0.00 | 0.0 ± 0.00 |
| B | Coleitriol | 6 | Topically | 0.2 mL/animal | 0.0 ± 0.00 | 0.0 ± 0.00 | 0.0 ± 0.00 | 0.0 ± 0.00 |
| | 0.01% | | | | | | | |
| C | Compound A | 6 | Topically | 0.2 mL/animal | 0.0 ± 0.00 | 1.0 ± 0.00 | 1.0 ± 0.00 | 1.2 ± 0.11 |
| | 1% | | | | | | | |
| D | Compound A | 6 | Topically | 0.2 mL/animal | 0.0 ± 0.00 | 0.0 ± 0.00 | 0.0 ± 0.00 | 0.0 ± 0.00 |
| | 0.1% | | | | | | | |

| | Assessment of erythema | | | | | | |
|---|---|---|---|---|---|---|---|
| Group | Day 5 | Day 6 | Dau 7 | Day 8 | Day 9 | Day 10 | Day 11 |
| A | 0.0 ± 0.00 | 0.0 ± 0.00 | 0.0 ± 0.00 | 0.0 ± 0.00 | 0.0 ± 0.00 | 0.0 ± 0.00 | 0.0 ± 0.00 |
| B | 0.0 ± 0.00 | 0.0 ± 0.00 | 0.0 ± 0.00 | 1.0 ± 0.00 | 1.5 ± 0.22 | 2.0 ± 0.20 | 2.0 ± 0.00 |
| C | 1.3 ± 0.17 | 1.5 ± 0.22 | 1.5 ± 0.22 | 1.2 ± 0.40 | 0.9 ± 0.15 | 0.9 ± 0.15 | 1.2 ± 0.17 |
| D | 0.0 ± 0.00 | 0.0 ± 0.00 | 0.0 ± 0.00 | 0.0 ± 0.00 | 0.0 ± 0.00 | 0.0 ± 0.00 | 0.0 ± 0.00 |

TABLE 8
Overall evaluation of body weight

| | | | Route of | Volume of | Body weight (g) | | | |
|---|---|---|---|---|---|---|---|---|
| Group | Treatment | n | administration | administration | Baseline | Day 2 | Day 3 | Day 4 |
| A | Control | 6 | Topically | 0.2 mL/animal | 0.0 ± 0.00 | 0.0 ± 0.00 | 0.0 ± 0.00 | 0.0 ± 0.00 |
| B | Coleitriol | 6 | Topically | 0.2 mL/animal | 0.0 ± 0.00 | 0.0 ± 0.00 | 0.0 ± 0.00 | 0.0 ± 0.00 |
| | 0.01% | | | | | | | |
| C | Compound A | 6 | Topically | 0.2 mL/animal | 0.0 ± 0.00 | 1.0 ± 0.00 | 1.0 ± 0.00 | 1.2 ± 0.11 |
| | 1% | | | | | | | |
| D | Compound A | 6 | Topically | 0.2 mL/animal | 0.0 ± 0.00 | 0.0 ± 0.00 | 0.0 ± 0.00 | 0.0 ± 0.00 |
| | 0.1% | | | | | | | |

| | Body weight (g) | | | | | | |
|---|---|---|---|---|---|---|---|
| Group | Day 5 | Day 6 | Dau 7 | Day 8 | Day 9 | Day 10 | Day 11 |
| A | 0.0 ± 0.00 | 0.0 ± 0.00 | 0.0 ± 0.00 | 0.0 ± 0.00 | 0.0 ± 0.00 | 0.0 ± 0.00 | 0.0 ± 0.00 |
| B | 0.0 ± 0.00 | 0.0 ± 0.00 | 0.0 ± 0.00 | 1.0 ± 0.00 | 1.5 ± 0.22 | 2.0 ± 0.20 | 2.0 ± 0.00 |
| C | 1.3 ± 0.17 | 1.5 ± 0.22 | 1.5 ± 0.22 | 1.2 ± 0.40 | 0.9 ± 0.15 | 0.9 ± 0.15 | 1.2 ± 0.17 |
| D | 0.0 ± 0.00 | 0.0 ± 0.00 | 0.0 ± 0.00 | 0.0 ± 0.00 | 0.0 ± 0.00 | 0.0 ± 0.00 | 0.0 ± 0.00 |

From the results obtained, it can be concluded that after topical application of 0.2 ml/animal of the reference product Calcitriol 0.01%, it was observed that the amount of corneous material extracted was clearly greater than that extracted in the other experimental groups. However, in this treatment group, at the specified dose, clear signs of toxicity were detected. This increase in corneous material that was susceptible to desquamation is described as a possible consequence of a terminal differentiation of keratinocytes in the epidermis.

After the group treated with the reference substance, the group that was administered compound A at 1% was the group from which the greatest amount of corneous material was extracted, presenting statistically significant differences in relation to the control group and the group treated with compound A at 0.1% (Student Newman Keuls, p<0.01), possibly due to an effect on the epidermis similar to that described for Calcitriol. Despite observing that the amount of corneous material extracted in this group was lower than that of the reference group, none of the animals presented signs of toxicity, which means that it is considered a safer dose than that of the Calcitriol 0.01% treatment.

The control group and the group treated with compound A at 0.1% presented values that were lower than those observed in the other groups, and it was considered that it had no effect on the animals' skin.

Three of the animals in the Calcitriol 0.01% group died before the end of the study, possibly due to toxic effects resulting from the repeated administration of the product.

From the studies that were carried out it can be concluded that compound A has clear beneficial effects for the treatment of psoriasis and can therefore be used for the treatment of said disorder in pharmaceutical compositions for topical use that can be administered in the form of a cream, gel or any other normal pharmaceutical form.

1. A chemical composition for treating skin disorders in humans, comprising:
a compound having a chemical formula of

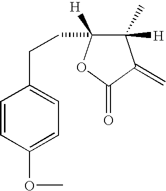

2. The chemical composition according to claim 1, wherein the skin disorder is psoriasis.

3. Pharmaceutical composition for topical use, comprising:
an active compound with a chemical formula 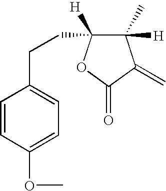
said active compound being in a proportion of between 0.1% and 5% w/w.

4. Pharmaceutical composition according to claim 3, being administered in solid or liquid form.

5. Pharmaceutical composition according to claim 4, being administered in the form of gel, cream, spray, foam, lotion or oil.
